(19) United States
(12) Patent Application Publication
(10) Pub. No.: US 2007/0093549 A1
(43) Pub. Date: Apr. 26, 2007

(54) Methods for preparation of ladostigil tartrate crystalline form A1

(76) Inventors: Judith Aronhime, Rehovot (IL); Eliezer Bahar, Tel-Aviv (IL); Anton Frenkel, Modi'in (IL); Ronen Gottesfeld, Netanya (IL); Tamas Koltai, Netanya (IL)

Correspondence Address: KENYON & KENYON LLP, ONE BROADWAY, NEW YORK, NY, 10004, US

(21) Appl. No.: 11/541,150

(22) Filed: Sep. 28, 2006

Related U.S. Application Data
(60) Provisional application No. 60/721,735, filed on Sep. 28, 2005.

(51) Int. Cl. A61K 31/325; C07C 261/02

(52) U.S. Cl. 514/490; 560/134

(57) ABSTRACT

Provided are processes for preparing crystalline ladostigil tartrate form A1.

### RELATED APPLICATIONS

This application claims the benefit of U.S. Provisional Application No. 60/721,735, filed Sep. 28, 2005.

### FIELD OF THE INVENTION

The invention relates to methods for the preparation of crystalline ladostigil tartrate.

### BACKGROUND OF THE INVENTION

Ladostigil is an active pharmaceutical ingredient which has shown to be effective in animal models of Alzheimer's disease. It contains a (R)-N-propargyl aminoindan moiety which is a monoamine oxidase type B inhibitor. It also contains a carbamate moiety which is effective as an acetylcholine esterase inhibitor. Ladostigil is disclosed in Weinstock, M. et al: J Neuronal Transm. (2000) [suppl]; 60: 157-169, Weinstock, M. et al: Development Research (2000); 50:216-222, Sterling J. et al: J. Med. Chem. 2002; 45:5260-5279, Weinstock M. et al: Psychopharmacology 2002; 160:318-324; and Yogev-Falach et al: FASEB J. 2002; October 16(12) :1674-1676.

The chemical name of ladostigil tartrate is carbamic acid, ethylmethyl-, (3R)-2,3-dihydro-3-(2-propynylamino)-1H-inden-5-yl ester, (2R,3R)-2,3-dihydroxybutanedioate (2:1). Its chemical structure is:

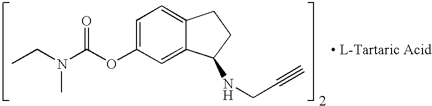

Ladostigil tartrate and a method for its preparation are disclosed in U.S. Pat. No. 6,303,650, hereby incorporated by reference. The '650 patent discloses the preparation of ladostigil tartrate by crystallization in isopropanol.

The occurrence of different crystal forms (polymorphism) is a property of some molecules and molecular complexes. A single molecule, or a salt complex, may give rise to a variety of solids having distinct physical properties like melting point, X-ray diffraction pattern, infrared absorption fingerprint and NMR spectrum. The crystalline form may give rise to thermal behavior different from that of the amorphous material or another crystalline form. Thermal behavior is measured in the laboratory by such techniques as capillary melting point, thermogravimetric analysis (“TGA”) and differential scanning calorimetry (“DSC”) and can be used to distinguish some polymorphic forms from others. The differences in the physical properties of different crystalline forms result from the orientation and intermolecular interactions of adjacent molecules (complexes) in the bulk solid.

Exemplary solid state physical properties include the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

One of the most important physical properties of pharmaceutical polymorphs is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment.

The ladostigil tartrate salt obtained in the '650 patent is reported to have a melting point of 143-145° C.

### SUMMARY OF THE INVENTION

One embodiment of the invention provides a process for preparing crystalline ladostigil tartrate characterized by an XRD pattern having peaks at 8.7, 13.9, and 17.4±0.2 degrees 2 theta, comprising the steps of:
(a) preparing a solution of ethyl-methyl-carbamic acid (R)-3-prop-2-ynylamino-indan-5-yl ester in ethanol, ethyl acetate, acetone, acetonitrile, diisopropylether or mixtures thereof;
(b) combining tartaric acid with the solution to form a precipitate;
(c) recovering the precipitate; and
(d) drying the precipitate to obtain the crystalline ladostigil tartrate.

Another embodiment of the invention provides a process for preparing crystalline ladostigil tartrate characterized by an XRD pattern having peaks at 8.7, 13.9, and 17.4±0.2 degrees 2 theta, comprising the steps of:
(a) preparing a solution of ladostigil tartrate in a C₁-C₄ alcohol, acetone, methyl ethyl ketone, tetrahydrofuran, acetonitrile, ethyl acetate, diisopropylether or mixtures thereof;
(b) precipitating the crystalline form; and
(c) recovering the crystalline form.

Yet another embodiment of the invention provides a process for preparing crystalline ladostigil tartrate characterized by an XRD pattern having peaks at 8.7, 13.9, and 17.4±0.2 degrees 2 theta, comprising the steps of:
(a) maintaining a heterogeneous mixture of ladostigil tartrate in acetate, ethyl acetate, dioxane or mixtures thereof; and
(b) recovering from the mixture the crystalline ladostigil tartrate.

Another embodiment of the invention provides a process for preparing crystalline ladostigil tartrate characterized by an XRD pattern having peaks at 8.7, 13.9, and 17.4±0.2 degrees 2 theta, comprising drying ladostigil tartrate form B or form C.

Another embodiment of the invention provides a process for preparing crystalline ladostigil tartrate characterized by an XRD pattern having peaks at 8.7, 13.9, and 17.4±0.2 degrees 2 theta, comprising heating ladostigil tartrate form B, form C, form F, or form H.

Another embodiment of the invention provides a pharmaceutical composition comprising a therapeutically effective amount of ladostigil tartrate characterized by an XRD pattern having peaks at 8.7, 13.9, and 17.4±0.2 degrees 2 theta, and a pharmaceutically acceptable carrier.

Another embodiment of the invention provides a process of preparing a pharmaceutical composition comprising the step of combining ladostigil tartrate form A1, or a solution prepared from crystalline ladostigil tartrate characterized by an XRD pattern having peaks at 8.7, 13.9, and 17.4±0.2 degrees 2 theta, with a pharmaceutically acceptable carrier.

Another embodiment of the invention provides a process of preparing a pharmaceutical composition comprising the steps of preparing crystalline ladostigil tartrate characterized by an XRD pattern having peaks at 8.7, 13.9, and 17.4±0.2 degrees 2 theta, according to the methods of the invention, and combining the ladostigil tartrate, or a solution prepared from the crystalline ladostigil tartrate, with a pharmaceutically acceptable carrier.

Another embodiment of the invention provides a method of treating Alzheimer's disease comprising administering to a human subject in need thereof a pharmaceutical composition comprising a therapeutically effective amount of crystalline ladostigil tartrate characterized by an XRD pattern having peaks at 8.7, 13.9, and 17.4±0.2 degrees 2 theta, prepared according to the methods of the invention, and a pharmaceutically acceptable carrier.

Another embodiment of the invention provides a method of treating a mammal in need of inhibition of the acetylcholine esterase enzyme comprising administering a pharmaceutical composition comprising a therapeutically effective amount of crystalline ladostigil tartrate characterized by an XRD pattern having peaks at 8.7, 13.9, and 17.4±0.2 degrees 2 theta, prepared according to the methods of the invention, and a pharmaceutically acceptable carrier, to the mammal.

Another embodiment of the invention provides a method of treating a mammal in need of inhibition of the monoamine oxidase type B enzyme comprising administering a pharmaceutical composition comprising a therapeutically effective amount of crystalline ladostigil tartrate characterized by an XRD pattern having peaks at 8.7, 13.9, and 17.4±0.2 degrees 2 theta, prepared according to the methods of the invention, and a pharmaceutically acceptable carrier, to the mammal.

### BRIEF DESCRIPTION OF THE DRAWINGS

FIG. 1 is a characteristic x-ray diffraction spectrum of ladostigil tartrate form A1.

FIG. 2 is a characteristic differential scanning calorimetric (DSC) thermogram of ladostigil tartrate form A1.

FIGS. 3a, 3b, and 3c are characteristic infrared (IR) spectra of ladostigil tartrate form A1.

FIGS. 4a, 4b, and 4c are characteristic Raman spectra of ladostigil tartrate form A1.

FIG. 5 is a characteristic x-ray diffraction spectrum of ladostigil tartrate Form B.

FIG. 6 is a characteristic differential scanning calorimetric (DSC) thermogram of ladostigil tartrate Form B.

FIG. 7 is a characteristic x-ray diffraction spectrum of ladostigil tartrate Form C.

FIG. 8 is a characteristic x-ray diffraction spectrum of ladostigil tartrate Form F.

FIG. 9 depicts characteristic data obtained from various techniques for ladostigil tartrate Form F.

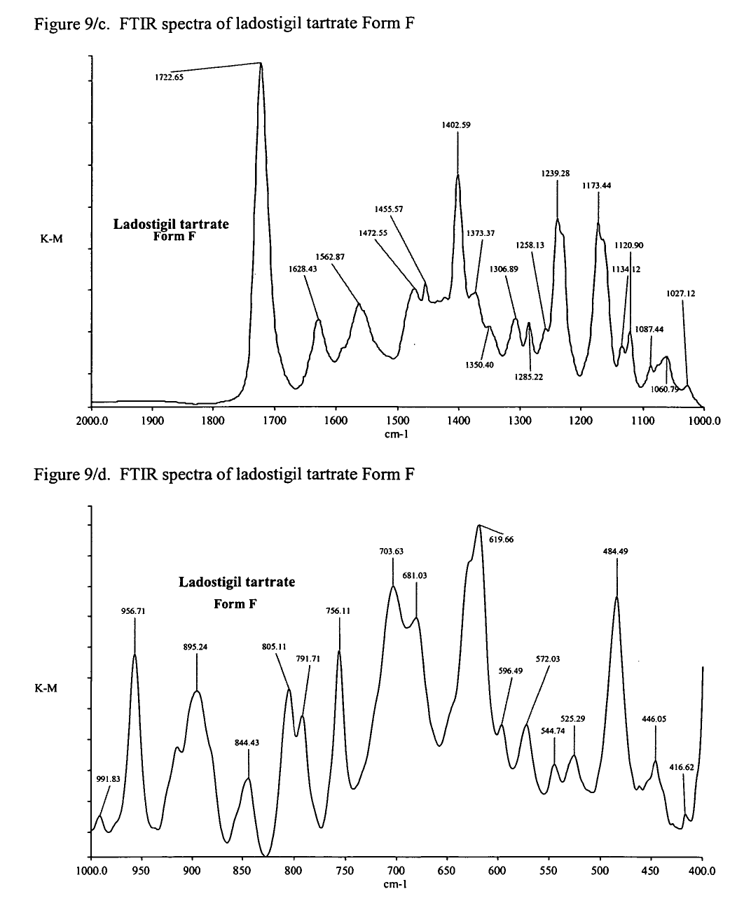

FIG. 10 is a characteristic DSC thermogram of ladostigil tartrate Form F.

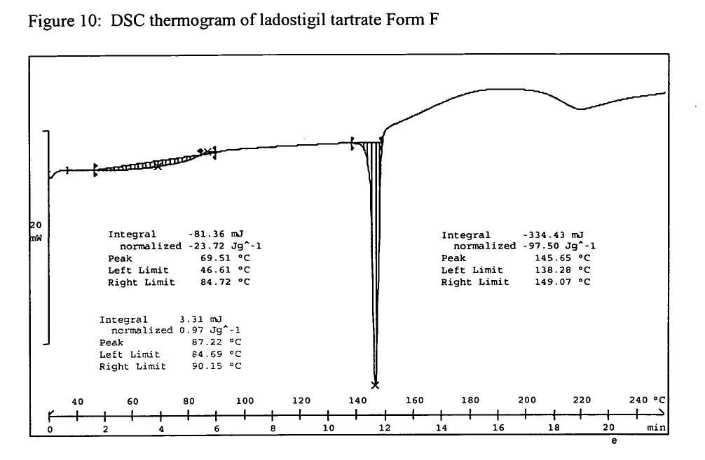

FIG. 11 is a characteristic x-ray diffraction spectrum of ladostigil tartrate Form H.

### DETAILED DESCRIPTION OF THE INVENTION

The following is a list of abbreviations as used herein.

| | |
|---|---|
| MAO | Monoamine oxidase |
| DSC | Differential scanning calorimetry |
| IR | Infrared |
| TGA | Thermogravimetric analysis |
| MeOH | Methanol |
| MEK | Methyl ethyl ketone |
| FTIR | Fourier transform infrared |
| XRD | X-ray diffraction |
| RH | Relative humidity |
| IPA | Isopropyl alcohol |
| EtOAc | Ethyl acetate |
| EtOH | Ethanol |
| KF | Karl Fischer |

As used herein, the term “vacuum” refers to a pressure below about 100 mm Hg.

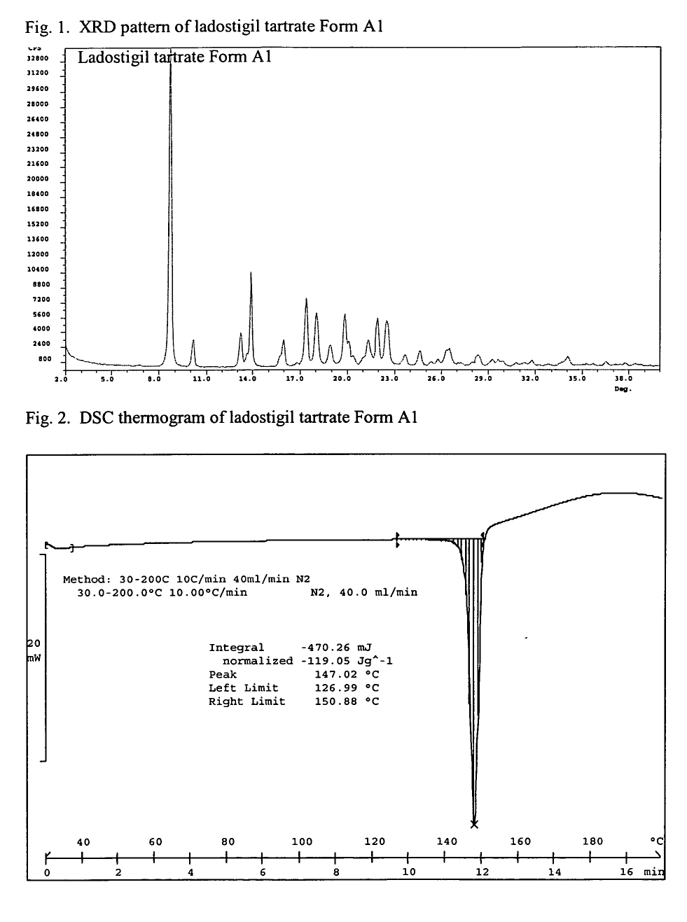

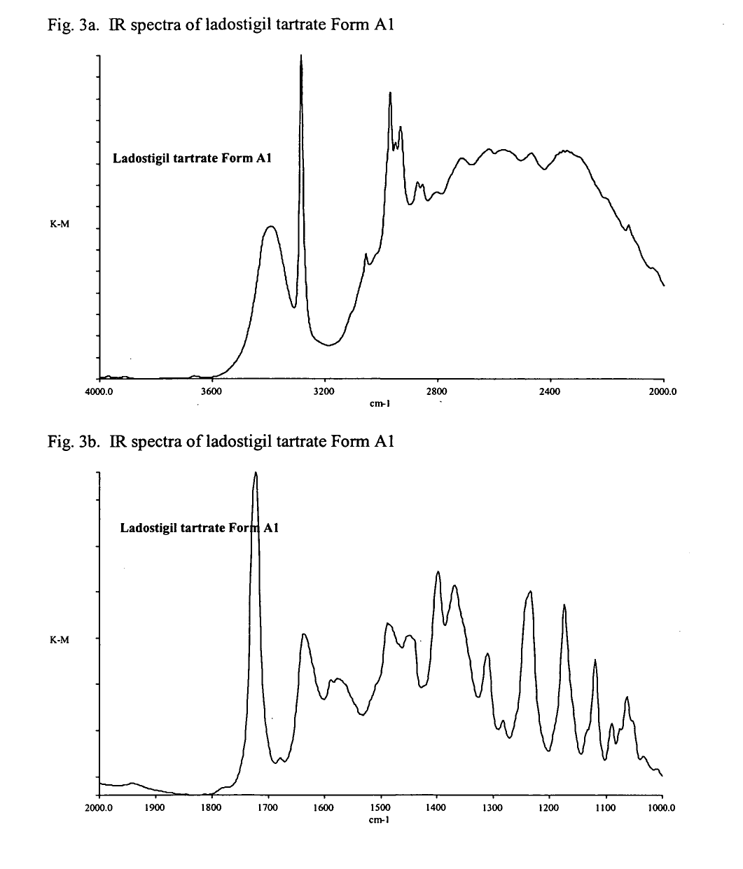

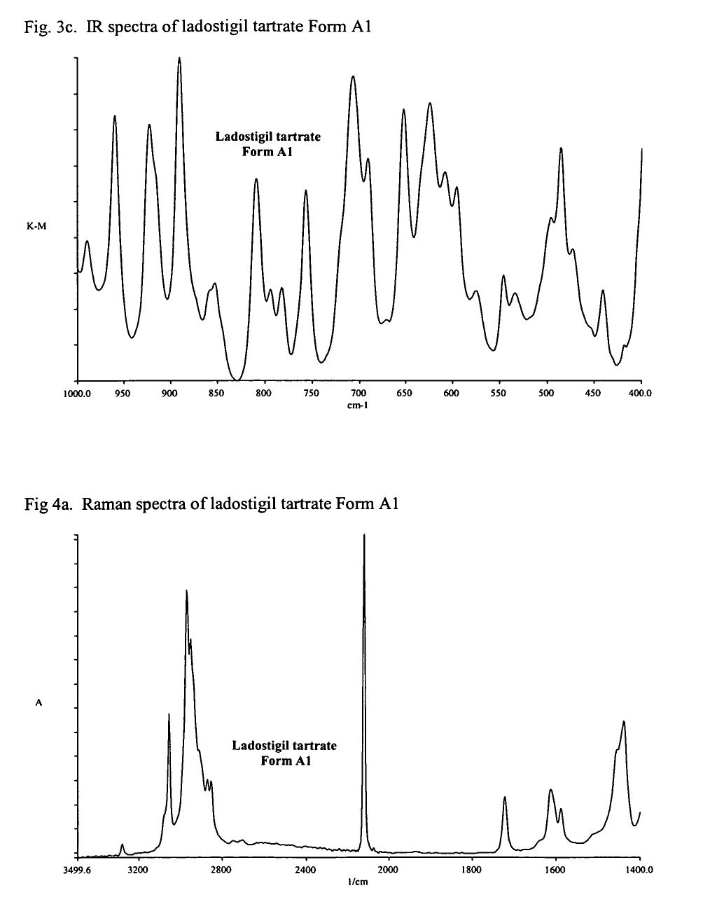

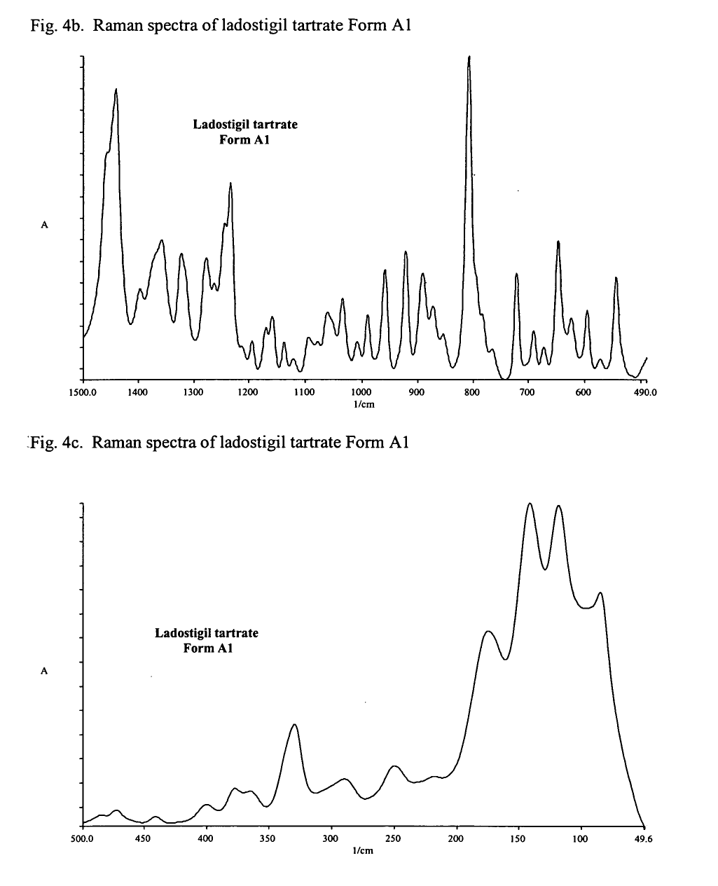

The invention encompasses processes for the preparation of crystalline ladostigil tartrate form A1. Ladostigil tartrate form A1 may be prepared by methods such as precipitation, crystallization, heating, drying, or slurrying. Crystalline Form A1 is characterized in FIGS. 1-4. Crystalline Form A1 is characterized by an XRD pattern having peaks at 8.7, 13.9, and 17.4±0.2 degrees 2 theta. Form A1 is further characterized by an XRD pattern having peaks at 19.8, 22.0, and 22.5±0.2 degrees 2 theta. The differential scanning calorimetric (DSC) thermogram of Form A1 is characterized by an endothermic peak at about 147° C. followed by a wide exothermic peak. The exothermic peak most probably represents decomposition of the compound. The water content of crystalline Form A1 is about 0.3% water by weight. The loss upon drying, as determined by TGA, is 0.3% by weight. Form A1 is anhydrous.

Formation of Ladostigil Tartrate Form A1 by Precipitation

In one embodiment, ladostigil tartrate form A1 is made by preparing a solution of ethyl-methyl-carbamic acid (R)-3-prop-2-ynylamino-indan-5-yl ester in an organic solvent; combining L-tartaric acid with the solution; forming a precipitate; recovering and drying the precipitate to obtain ladostigil tartrate form A1.

Suitable organic solvents include at least one of ethanol, ethyl acetate, acetone, acetonitrile, and diisopropylether. The solution may further include at least one of toluene or dioxane. The solvent may be present in any amount sufficient to precipitate ladostigil tartrate form A1. Preferably, the solvent is present in an amount of about 10 ml/g of the ethyl-methyl-carbamic acid (R)-3-prop-2-ynylamino-indan-5-yl ester. In a preferred embodiment, the solvent is ethanol combined with at least one of toluene, acetone, ethyl acetate, or dioxane. In a more preferred embodiment, the toluene, acetone, ethyl acetate, or dioxane is present in an amount of about 1% to about 10% relative to the weight of ethanol, more preferably about 2% to about 6%. Other preferred solvent ratios are exemplified in Example 6.

The L-tartaric acid may be a solid, or it may be in solution. Preferably, if in solution, the L-tartaric solution is warmed. After addition of the L-tartaric acid, the resulting mixture may be heated, preferably at reflux temperature, or at a temperature below the boiling point of the solvent. The mixture is preferably heated for about ¼ hours to about 2 hours, more preferably for about ½ hour to about 1 hour.

The mixture may be cooled to form a precipitate. Preferably, the mixture is cooled to about 30° to about 0° C., more preferably to about 5° C. Cooling is preferably done gradually to form the precipitate. For example, the mixture may be cooled over a period of about 2 hours to about 25 hours. Preferably, the mixture is maintained at the cooling temperature for about 1 hour to about 5 hours, more preferably about 1 hour to about 2 hours.

The precipitate may be collected by filtration. The precipitate may be air dried at room temperature, or it may be dried at elevated temperatures. In one embodiment, the precipitate is dried under vacuum at room temperature. In another embodiment, the precipitate is dried under vacuum at a temperature of about 50° C. to about 90° C., more preferably at about 50° C. The precipitate is preferably dried until constant weight. In a preferred embodiment, the precipitate is dried for about 20 hours to about 30 hours, more preferably for about 24 hours to about 26 hours. One of ordinary skill in the art would understand that the amount of time the precipitate is dried to obtain form A1 will depend on the weight of the precipitate.

Formation of Ladostigil Tartrate Form A1 by Re-Crystallization

In another embodiment, ladostigil tartrate form A1 is made by preparing a solution of ladostigil tartrate in a solvent; crystallizing Form A from the solution; and recovering the precipitate as ladostigil tartrate form A1.

Suitable solvents for preparing a solution of ladostigil tartrate include at least one of a C₁-C₄ alcohol, acetone, methyl ethyl ketone, tetrahydrofuran, acetonitrile, ethyl acetate, or diisopropylether. C₁-C₄ alcohols include, for example, methanol, ethanol, propanol, butanol, and isobutanol. The solution may further include at least one of water or acetic acid. The solvent may be present in any amount sufficient to crystallize ladostigil tartrate form A1. Preferably, the solvent is present in an amount of about 1 ml/g to about 45 ml/g of the ladostigil tartrate, more preferably about 1.3 ml/g to about 43 ml/g of the ladostigil tartrate. In one preferred embodiment, the solvent is ethanol combined with at least one of water, acetic acid, or methanol. In a more preferred embodiment, the water, acetic acid, or methanol is present in an amount of about 1% to about 10% of the ethanol by volume, more preferably about 2.5% to about 7.5%. Other preferred solvent ratios are exemplified in the Examples section.

The solution may be heated, preferably at reflux temperature, or at a temperature below the boiling point of the solvent. The mixture is preferably heated for about ¼ hours to about 2 hours, more preferably for about ½ hour to about 1 hour.

The solution may be cooled to form a precipitate. Preferably, the solution is cooled to about 30° C. to about 0° C., more preferably to about 5° C. The solution is preferably cooled to the cooling temperature gradually, for example, in about 4 hours to about 25 hours, more preferably in about 5 hours to about 23 hours. Preferably, the solution is maintained at the cooling temperature for about 1 hour to about 5 hours, more for preferably about 1 hour to about 2 hours. The solution may also be seeded to form a precipitate.

The precipitate may be collected by filtration. In one embodiment, the precipitate is dried under vacuum in a microwave oven for about 10 minutes. In another embodiment, the precipitate is dried under vacuum at ambient temperature. In another embodiment, the precipitate is dried under vacuum at a temperature of about 50° C. to about 90° C., more preferably at about 50° C. The precipitate is preferably dried until constant weight. In a preferred embodiment, the precipitate is dried for about 10 hours to about 30 hours, more preferably for about 14 hours to about 24 hours. One of ordinary skill in the art would understand that the amount of time the precipitate is dried to obtain form A1 will depend on the weight of the precipitate.

Formation of Ladostigil Tartrate form A1 by Slurrying

In another embodiment, ladostigil tartrate form A1 is made by maintaining a heterogeneous mixture of ladostigil tartrate in a solvent; and recovering from the mixture the ladostigil tartrate form A1.

Suitable solvents include at least one of isobutyl acetate, ethyl acetate, or dioxane. Preferably, the ladostigil tartrate used in the heterogeneous mixture is ladostigil tartrate form 1A or ladostigil tartrate form C.

The heterogeneous mixture may be maintained at a temperature of about 0° C. to about 30° C., preferably at about 8° C. to about 28° C. The heterogeneous mixture may also be heated, and if heated, preferably is heated at a temperature of about 50° C. to about 80° C., more preferably at about 62° C. to about 70° C.

The ladostigil tartrate form A1 may be collected by filtration. Preferably, the ladostigil tartrate is dried under vacuum at a temperature of about 50° C. to about 90° C., more preferably at about 50° C. or about 80° C. The ladostigil tartrate is preferably dried until constant weight.

Formation of Form A1 by Drying

In another embodiment, ladostigil tartrate form A1 is prepared by drying ladostigil tartrate form B or form C. Preferably, the ladostigil tartrate form B or C is dried under vacuum, and preferably at a temperature of about 50° C. to about 90° C.

Upon heating, form C transforms to form B, which may subsequently transform into form A1. The dried product may also contain form A and/or form E.

Formation of Ladostigil Tartrate Form A1 by Heating

In another embodiment, ladostigil tartrate form A1 is prepared by heating ladostigil tartrate form B, form C, form F, or form H. Preferably, the ladostigil tartrate is heated under vacuum. Preferably, the ladostigil tartrate is heated at a temperature of about 50° C. to about 90° C., more preferably at about 50° C. or about 80° C.

Other Ladostigil Tartrate Forms

Ladostigil tartrate forms B, C, F, and H are characterized by X-Ray peaks as described below in Table 1. Ladostigil tartrate forms A1, B, and C are also characterized by IP peaks illustrated below in Table 2. The preparation of ladostigil tartrate forms B, C, F, H, and G is described in the Examples herein, and in co-pending application No. 60/721,714. Application No. 60/721,714, and the specific processes for preparing ladostigil tartrate forms B, C, F, H, and G that are described therein, are incorporated herein by reference.

Form B

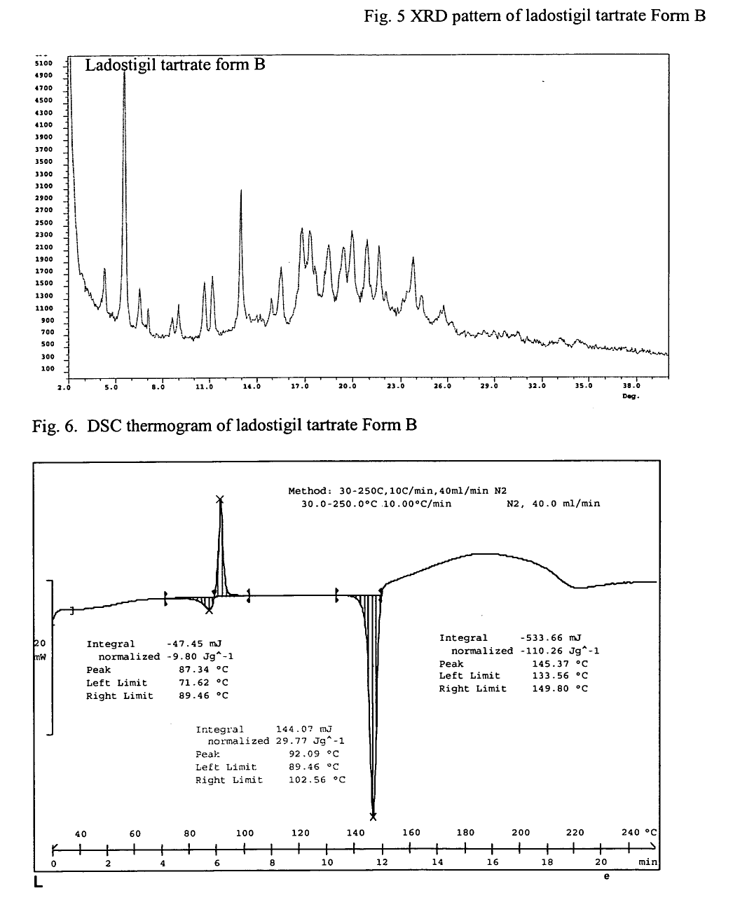

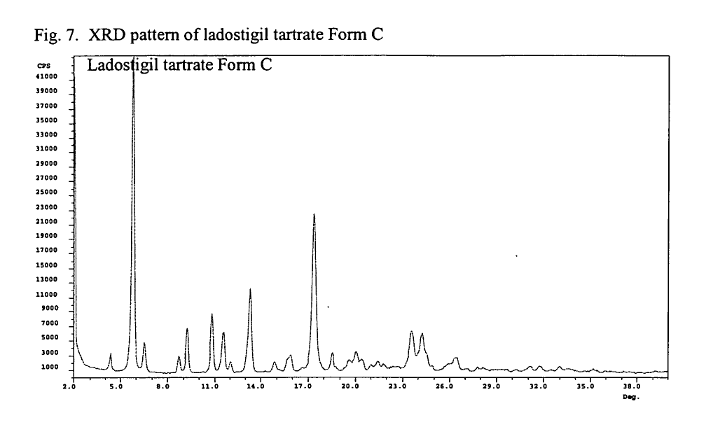

Ladostigil tartrate form B has an X-ray powder diffractogram as substantially depicted in FIG. 5. The DSC thermogram of form B is shown in FIG. 6. The DSC thermogram of form B shows a small endothermic peak followed by an exothermic peak at about 87° C. and 92° C. attributed to the conversion of form B into form A1. These two peaks are followed by the known endothermic peak of Form A1 at about 147° C., followed by an exothermic peak at about 190° C. which correspond, respectively, to the melting and decomposition of form A1. Form B contains about 1% loss on as drying as determined by TGA.

Ladostigil tartrate form A1 may be prepared by heating or drying form B.

Form C

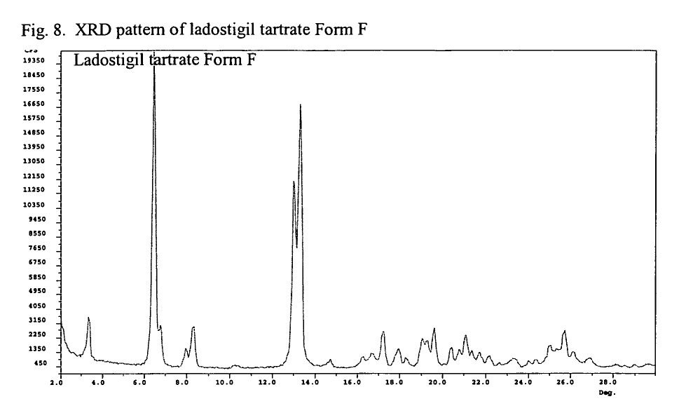

Ladostigil tartrate form C has an X-ray powder diffractogram as substantially depicted in FIG. 7. The DSC thermogram of form C shows small endothermic peaks probably due to the evaporation of solvents from the sample. From an analysis of heating of Form C at 50° C., it can be seen that with the evaporation of solvent form C transforms to form B. These desolvation peaks are followed by an endothermic and exothermic peak at about 87 and 92° C. respectively, due to the conversion of Form B into form A1. These two peaks are followed by the known endothermic peak of Form A1 at about 147° C. followed by an exothermic peak at about 190° C., which correspond respectively to the melting and decomposition of form A1.

TGA analysis of the studied form C shows a weight loss step of about 40% w/w due to the removal of solvents. Water content by Karl Fisher analysis is about 2% w/w. From this disparity in the difference of weight loss and water content we deduce that most of the moisture content is due to the presence of ethanol.

Ladostigil tartrate form A1 may be prepared by heating or drying form C.

Form F

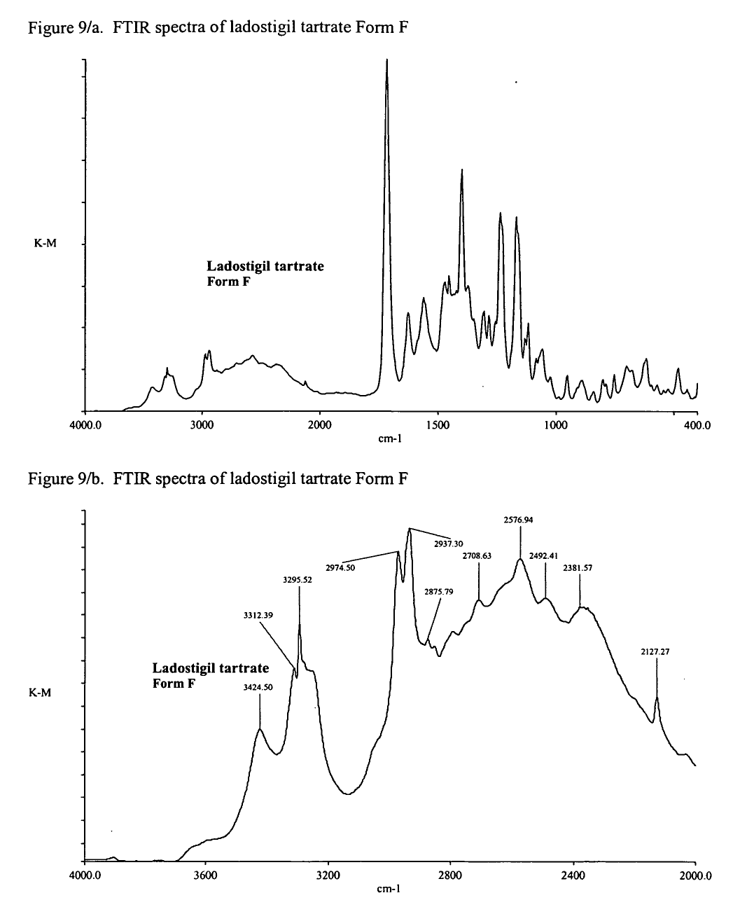

Ladostigil tartrate form F has an X-ray powder diffractogram as substantially depicted in FIG. 8. Form F may be characterized by an FTIR spectrum with characteristic absorption bands at about 3425, 3296, 1628, 1403 cm⁻¹. Form F may be characterized by a DSC thermogram with a broad endothermic peak at 70° C., an exothermic peak at about 90° C. and a final melting endotherm 145° C. with decomposition at 170° C.

Ladostigil tartrate form A1 may be prepared by heating form F.

Form H

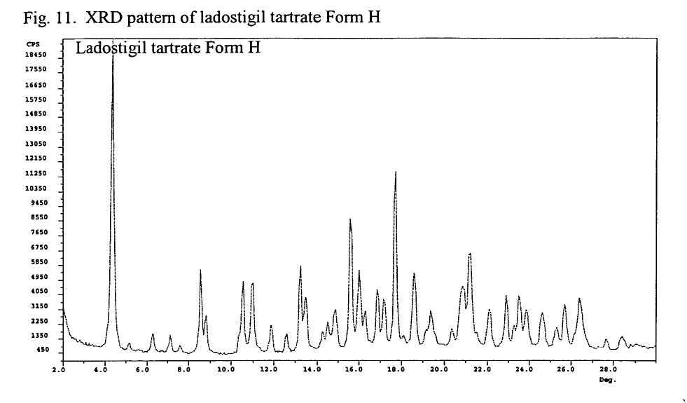

Ladostigil tartrate form H has an X-ray powder diffractogram as substantially depicted in FIG. 11. Ladostigil tartrate form A1 may be prepared by heating form F.

The starting material used in the processes of the invention may be any crystalline or amorphous form of ladostigil tartrate, including various solvates and hydrates. With crystallization processes, the crystalline form of the starting material does not usually affect the final result. With slurrying, the final product may vary depending on the starting material. One of skill in the art would appreciate the manipulation of the starting material within skill in the art to obtain a desirable form with slurry. The invention is not limited to the starting form used for slurry unless such form is essential for obtaining another form.

The processes of the invention encompass crystallization out of a particular solvent, i.e., obtaining a solid material from a solution. One skilled in the art would appreciate that the conditions concerning crystallization may be modified without affecting the ladostigil tartrate polymorph obtained. For example, when mixing a solid in a solvent to form a solution, warming of the mixture may be necessary to completely dissolve the starting material. If warming does not clarify the mixture, the mixture may be diluted or filtered. To filter, the hot mixture may be passed through paper, glass fiber or other membrane material, or a clarifying agent such as celite. Depending upon the equipment used and the concentration and temperature of the solution, the filtration apparatus may need to be preheated to avoid premature crystallization.

The conditions may also be changed to induce precipitation. A preferred way of inducing precipitation is to reduce the solubility of ladostigil tartrate in the solvent. The solubility may be reduced, for example, by cooling the solvent. Precipitation may also be induced by adding a co-solvent to the solution. Preferably, the co-solvent is selected from the group consisting of polar and non-polar aprotic solvents. More preferably, the co-solvent is toluene, acetone, ethyl acetate, or dioxane. The cosolvent is preferably added in an amount of about 1% to 20% by weight of the solvent present in the solution of ladostigil tartrate.

Another way of accelerating crystallization is by seeding with a ladostigil tartrate crystal. Crystallization may occur spontaneously without any inducement.

The invention also provides a pharmaceutical composition comprising ladostigil tartrate form A1, and at least one pharmaceutically acceptable excipient. The invention further provides a pharmaceutical composition prepared from ladostigil tartrate form A1. The invention further encompasses a process for preparing a pharmaceutical composition comprising combining ladostigil tartrate form A1 with at least one pharmaceutically acceptable excipient.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Pharmaceutical formulations of the invention contain the above disclosed polymorphic forms ladostigil tartrate. The pharmaceutical composition may contain only a single form of ladostigil tartrate, or a mixture of various forms of ladostigil tartrate, with or without amorphous form. In addition to the active ingredient(s), the pharmaceutical compositions of the invention may contain one or more excipients or adjuvants. Selection of excipients and the amounts to use may be readily determined by the formulation scientist based upon experience and consideration of standard procedures and reference works in the field.

Diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions of the invention, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the invention, a liquid composition may also contain a buffer such as guconic acid, lactic acid, citric acid or acetic acid, sodium guconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granulates that were described with reference to tableting, however, they are not subjected to a final tableting step.

When preparing injectable (parenteral) pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added.

The dosage used is preferably from about 0.5 mg to about 500 mg of ladostigil tartrate, more preferably about 20 to about 100 mg. The pharmaceutical compositions of the present invention are used to treat Alzheimer's disease in a mammal such as a human in need thereof.

Also provided is a method of treating a mammal in need of inhibition of the acetylcholine esterase enzyme comprising administering a pharmaceutical composition prepared from ladostigil tartrate form A1 to the mammal. Also provided is a method of treating a mammal in need of inhibition of the monoamine oxidase type B enzyme comprising administering a pharmaceutical composition prepared from ladostigil tartrate form A1 to the mammal.

TABLE 1
The following table discloses characteristic X-Ray diffraction
peaks for crystalline forms of ladostigil tartrate. The more
characteristic peaks are exemplified in bold.

| Form A1 | Form B | Form C | Form F | Form H |
|---|---|---|---|---|
| 8.7 | 4.3 | 4.3 | 3.3 | 4.4 |
| 10.2 | | | | 8.5 |
| 13.2 | 5.6 | 5.8 | 6.4 | 10.5 |
| 13.9 | 6.5 | 6.5 | 8.3 | 11.0 |
| 16.0 | 10.6 | 9.3 | 13.0 | 13.2 |
| 17.4 | 11.2 | | 13.3 | 15.6 |
| 18.0 | 13.0 | 10.8 | 17.2 | 16.0 |
| | 15.5 | 11.6 | | 16.8 |
| 18.9 | 16.8 | | | 17.2 |
| | 17.4 | 13.3 | 19.6 | 17.7 |
| 19.8 | 18.5 | 17.4 | 21.0 | 18.6 |
| 20.0 | 19.4 | | | 19.4 |
| | | | | 20.8 |
| | | | | 22.1 |
| | | | | 23.5 |
| 21.3 | 19.9 | 18.5 | 25.7 | 23.8 |
| 22.0 | 20.9 | 20.0 | | 21.2 |
| 22.5 | 21.6 | 23.6 | | 22.9 |
| 24.6 | 23.8 | 24.3 | | 26.3 |

TABLE 2

| Form A and A1 | Form B | Form C |
|---|---|---|
| 3388.0* | | 3901.4* |
| 3284.9* | 3290.5* | 3309.4* |
| 3056.1 | | |
| 2970.0* | 2973.5 | 2974.3 |
| 2953.2 | | |
| 2934.0 | 2938.3 | 2936.8 |
| 2873.1 | | |
| 2855.4 | | |
| 2805.3 | | |
| 2711.5 | | 2704.7 |
| 2620.8 | 2576.0 | |
| 2471.0 | | |
| 2351.9 | | |
| 2126.5 | | 2126.5 |
| 1722.8* | 1716.7 | 1716.8 |
| 1636.3* | 1626.8* | 1565.4 |
| 1487.7* | 1562.9 | 1476.0 |
| 1447.2* | 1475.3 | |
| 1398.5 | 1454.2 | 1401.4 |
| 1368.4* | 1401.6 | 1338.1* |
| 1309.0* | 1306.1 | 1305.6 |
| 1233.6* | 1285.6 | 1287.0 |
| 1174.3* | 1238.7* | 1261.0* |
| 1119.8 | 1166.1* | 1238.8 |
| 1090.0 | 1121.0 | 1169.2* |
| 1063.4 | 1087.2 | 1134.9* |
| 989.3* | 1064.0 | 1076.7* |
| 959.2 | 1027.7 | 1067.4* |
| 922.0* | 957.2 | 1027.5 |
| 890.2* | 892.9 | 957.6 |
| 852.4* | 844.2 | 903.3* |
| 808.9* | 804.5 | 843.4 |
| 794.1 | | 803.6 |
| 781.3* | | 790.9* |
| 757.0 | 755.6 | 755.6 |
| 707.7* | 700.9* | |
| 692.3* | | 680.5* |
| 653.6* | | |
| 624.8* | 632.4* | 619.9* |
| 607.1* | 617.4* | |
| 595.8 | 595.8 | 595.8 |
| 573.5 | | 571.4 |
| 545.6 | | |
| 533.6* | | 524.1 |
| 485.8 | 485.2 | 485.2 |
| 473.6 | | 446.5 |
| 441.0 | | 416.7 |

*More characteristic peaks.

Having described the invention with reference to certain preferred embodiments, other embodiments will become apparent to one skilled in the art from consideration of the specification. The invention is further described by reference to the following examples describing in detail the polymorph forms and processes for making them. It will be apparent to those skilled in the art that many modifications, both to materials and methods, may be practiced without departing from the scope of the invention.

### EXAMPLES

X-Ray powder diffraction data were obtained by a SCINTAG powder X-Ray diffractometer model X'TRA equipped with a solid state detector. Copper radiation of 1.5418 Å was used. A round aluminum sample holder with round zero background quartz plate, with cavity of 25 (diameter)*0.5 (depth) mm was used.

DSC analysis was done using a Mettler 821 Star. The weight of the samples was about 5 mg. The samples were scanned at a rate of 10° C./min from 30° C. to 320° C. The oven was constantly purged with nitrogen gas at a flow rate of 40 ml/min. Standard 40 μl aluminum crucibles covered by lids with 3 holes were used.

TGA analysis was done using a Mettler M3 meter. The weight of the samples was about 10 mg. The samples were scanned at a rate of 10° C./min from 25° C. to 200° C. The oven was constantly purged with nitrogen gas at a flow rate of 40 ml/min. Standard 70 μl alumina crucibles covered by lids with 1 hole were used.

IR analysis was performed using a Perkin Elmer “Spectrum One” FT-IR spectrometer in DRIFTt mode. The samples in the 4000-400 cm^{31 1} interval were scanned 16 times with 4.0 cm⁻¹ resolution.

Raman spectroscopy was performed on Bruker RFS-100/S Raman spectrometer. The samples in the 3500-50 cm⁻¹ interval were scanned 100 times with 4.0 cm⁻¹ resolution. Other parameters were set as follows:

| Aperture Setting | 10.0 | mm |
|---|---|---|
| Low Pass Filter 16; | 1 | KHz |
| Source Setting Laser; 9394.0 cm −1; | 1600 | mW |
| Raman Laser Power(mW) | 500 | |
| Scanner Velocity 5.0; | 4 | KHz |

The water content of ladostigil tartrate was measured by the methods known in the art, such Karl Fischer analysis.

Polymorph A1

### Example 1

### Preparation of Ladostigil Tartrate in Isopropanol

To a stirred solution of Ethyl-methyl-carbamic acid (R)-3-prop-2-ynylamino-indan-5-yl ester (100% assay) (25 g, 0.092 mole, 1 eq.) in isopropanol (240 ml) a warm solution of L-tartaric acid (7.2 g, 0.048 mole, 0.525 eq.) in isopropanol (27 ml) was added. The mixture was heated to complete dissolution and cooled gradually to 10° C. and maintained at this temperature for 2 hours. The product was collected by filtration washed with 2×33 ml cold isopropanol and portions of wet material were dried in a vacuum oven at 50, 60, 70, 80, 90° C. until constant weight was achieved. The material was sampled after one, two and three hours of drying.

The wet material was determined to be polymorph A1 with low crystallinity. The material was dried at 50, 60, 70, 80, 90° C. under vacuum. The material was sampled after one, two and three hours, and was determined to be polymorph A1.

### Example 2

### Re-Crystallization of Ladostigil Tartrate from Isopropanol

Ladostigil tartrate (42.2 g) was dissolved in isopropanol (420 ml) by heating the solution to 70° C. The mixture was cooled to 2° C. gradually for 6 hours. The product was collected by filtration and washed with 40 ml cold isopropanol. The sample was divided into parts. One part was dried at 25° C. under vacuum until a constant weight was achieved. One part was dried in a vacuum oven at 55-60° C. until a constant weight was achieved. Another part was dried for 2 hours in a vacuum oven at 80° C.

The wet material was determined to be polymorph A1.

The wet material dried at 25° C. under vacuum was determined to be polymorph A1.

The material dried at 50-60° C. under vacuum was polymorph A1.

The material dried at 80° C. under vacuum was polymorph A1.

### Example 2a

Ladostigil tartrate was crystallized from 3 volumes isopropanol as described above, and dried at 25° C. or at 80° C. The resulting product was determined to be polymorph A1.

Ladostigil tartrate was crystallized from 5 volumes isopropanol as described above and dried at 25° C. or at 80° C. The resulting product was determined to be polymorph A1.

### Example 3

### Preparation of Ladostigil Tartrate in Ethanol

To a stirred solution of Ethyl-methyl-carbamic acid (R)-3-prop-2-ynylamino-indan-5-yl ester (200 g, 0.734 mole, 1 eq.) in absolute ethanol (1000 ml, 790 gram) a warm solution of L-tartaric acid (58 g, 0.386 mole, 0.525 eq.) in absolute ethanol (800 ml, 630 gram) was added. The residue of tartaric acid in the vessel was washed with 100 ml (80 gram) absolute ethanol which was added to the reaction mixture. The mixture was heated to 58-62° C. and filtered if necessary, the filtration system was washed with 100 ml (80 gram) absolute ethanol. The solution was returned to the reactor and cooled to 0-5° C. gradually for 3-3.5 hours. The product was collected by filtration and washed with 150 ml cold ethanol and dried in a vacuum oven first at 25° C. under vacuum until a constant weight was obtained, then at 50° C. until constant weight, then at 80° C. to yield 210 g ladostigil tartrate (theoretical yield 87%).

The wet material was determined to be polymorph C.

The material dried at 25° C. under vacuum was determined to be polymorph A1.

The material dried at 50° C. under vacuum for 12 hours was determined to be polymorph B.

The material dried at 50° C. under vacuum for 12 hours, then at 80° C. under vacuum for 3 hours is a mixture of polymorphs consisting mainly of form A1 and a small amount of form E. If dried for a longer time, only polymorph A1 is obtained.

### Example 4a

### Re-Crystallization of Ladostigil Tartrate in 5 Volumes Ethanol

Ladostigil tartrate (50 g) was dissolved in ethanol (250 ml, 5 vol.) by heating the solution to 55-62° C. The mixture was cooled to 0-5° C. gradually for 4.5 hours and maintained at the same temperature for 2.5 hours. The product was collected by filtration washed with 30 ml cold ethanol.

The wet material obtained was polymorph C.

One part of the wet material was dried at 60° C. under vacuum for 14 hours, giving polymorph A.

One part of the wet material was dried at 60° C. under vacuum for 14 hours, then at 80° C. under vacuum for 3 hours, giving a mixture of polymorphs A1 and E, primarily polymorph E.

In another experiment, polymorph C was dried under vacuum at 50° C. for 20 hours and polymorph B was obtained. Further drying of the same material under vacuum at 80° C. for 3 hours resulted in a mixture of polymorphs A1 and E, primarily form E.

### Example 4b

### Re-Crystallization of Ladostigil Tartrate in 10 Volumes Ethanol

Ladostigil tartrate (50 g) was dissolved in ethanol (500 ml, 10 vol.) by heating the solution to 45-58° C. The mixture was cooled to 0-5° C. gradually for 4.5 hours and was maintained at the same temperature for 2.5 hours. The product was collected by filtration and washed with 30 ml cold ethanol.

The wet material was determined to be polymorph C.

One part of the material was dried at 60-70° C. under vacuum for 14 hours, and was determined to be polymorph A. Upon further drying for 3 hours under vacuum at 80° C., a mixture of polymorphs A1, E, and B was obtained, primarily polymorph A1.

One part of the wet material was dried at 80° C. under vacuum for 3 hours, giving a mixture of polymorphs A1+E.

Another part of the wet material was dried at 90° C. under vacuum for 3 hours. A mixture of polymorphs A1 and E was obtained, primarily polymorph A1.

### Example 5a

### Re-Crystallization of Ladostigil Tartrate in 1.3 Volumes Methanol

Ladostigil tartrate (33.2 g) was dissolved in methanol (43 ml, 1.3 vol.) by heating the solution to 40° C. The solution was seeded at 36° C. and cooled to 5° C. gradually over 20 hours. The product was collected by filtration and washed with 20 ml cold methanol.

The wet material was determined to be polymorph A1.

One part of the wet material was dried at 25° C. under vacuum, giving polymorph A1.

Another part of the wet material was dried at 80° C. under vacuum, giving polymorph A1.

### Example 5b

### Re-Crystallization of Ladostigil Tartrate in 10 Volumes n-Propanol

Ladostigil tartrate (20.2 g) was dissolved in n-propanol (200 ml, 10 vol.) by heating the solution to 55° C. The solution was seeded at 49° C. and cooled to 2° C. gradually over 12 hours. The product was collected by filtration and washed with 40 ml cold n-propanol.

One part of the wet material was dried at 25° C. under vacuum, giving polymorph A1.

Another part of the wet material was dried at 80° C. under vacuum, giving polymorph A1.

### Example 5c

### Re-Crystallization of Ladostigil Tartrate in 10 Volumes n-Butanol

Ladostigil tartrate (15.1 g) was dissolved in n-butanol (151 ml, 10 vol.) by heating the solution to 64° C. The solution was seeded at 62° C. and cooled to 3° C. gradually over 12 hours. The product was collected by filtration and washed with 20 ml cold n-butanol.

The wet material was determined to be polymorph A1.

One part of the wet material was dried at 25° C. under vacuum, giving polymorph A1.

Another part of the wet material was dried at 80° C. under vacuum, giving polymorph A1.

### Example 5d

### Re-Crystallization of Ladostigil Tartrate in 10 Volumes Iso-Butanol

Ladostigil tartrate (15.6 g) was dissolved in iso-butanol (156 ml, 10 vol.) by heating the solution to 69° C. The solution was seeded at 69° C. and was cooled to 3° C. gradually over 7 hours. The product was collected by filtration and washed with cold iso-butanol.

The wet material was determined to be polymorph A1.

One part of the wet material was dried at 25° C. under vacuum, giving polymorph A1.

Another part of the wet material was dried at 80° C. under vacuum, giving polymorph A1.

### Example 5e

### Re-Crystallization of Ladostigil Tartrate in 10 Volumes Ethanol and Drying Using Microwave

Ladostigil tartrate (114 g) was dissolved in ethanol (1140 ml, 10 vol.) by heating the solution to reflux. The mixture was cooled to 20° C. gradually, then to 5° C., and was maintained at the same temperature for 1 hour. The product was collected by filtration and washed with 2×60 ml cold ethanol.

The material was dried in a microwave oven (100 watt) under vacuum (60-100 mbar) for 9 minutes, and was determined to be polymorph A1.

### Example 5f

### Preparation of Ladostigil Tartrate in 10 Volumes of Ethanol Containing 6% Toluene as a Co-Solvent

To a stirred solution of Ethyl-methyl-carbamic acid (R)-3-prop-2-ynylamino-indan-5-yl ester (59.6 g) in absolute ethanol (253 ml, 200 gram) a warm solution of L-tartaric acid (17.2 g) in absolute ethanol (190 ml, 150 gram) was added. The residue of tartaric acid in the vessel was washed with 63 ml (50 gram) absolute ethanol and was poured into the reaction mixture. The mixture was heated to 58-62° C. and filtered, and the filtration system was washed with 90 ml (71 gram) absolute ethanol. The resulting solution (“solution A”) was used in the co-solvent salt preparation experiments.

Toluene 6% relative to ethanol weight was added to solution A and the solution was cooled in a controlled manner from 58° C. to 2° C. for 17 hours.

The wet material was collected by filtration and washed with cold ethanol. The wet material was determined to be polymorph C with low crystallinity.

One part of the wet material was dried at 55° C. under vacuum for 19 hours, giving polymorph B.

Another part of the wet material was dried at 55° C. under vacuum for 19 hours then at 80° C. under vacuum for 7 hours, giving a mixture of polymorphs A1 and E, primarily form A1.

### Example 5g

### Preparation of Ladostigil Tartrate in 10 Volumes of Ethanol Containing 2% Acetone as Co-Solvent

To the ethanolic solution A prepared as in example 5f, acetone 2% relative to ethanol weight was added and the solution was cooled in a controlled manner from 60° C. to 2° C. for 20 hours.

The wet material was determined to be polymorph C.

One part of the wet material was dried at 55° C. under vacuum for 18 hours, giving polymorph B.

Another part of the wet material was dried at 55° C. under vacuum for 18 hours, then at 80° C. under vacuum for 7 hours, giving a mixture of polymorphs A1 and E, primarily A1.

### Example 5h

### Preparation of Ladostigil Tartrate in 10 Volumes of Ethanol Containing 2.25% Ethyl Acetate as Co-Solvent

To the ethanolic solution A prepared as described in example 5f, ethyl acetate 2.25% relative to ethanol weight was added and the solution was cooled in a controlled manner from 60° C. to 2° C. for 23 hours.

One part of the wet material was determined to be polymorph C with low crystallinity.

Another part of the wet material was dried at 55° C. under vacuum for 17 hours, giving polymorph B.

Another part of the wet material was dried at 55° C. under vacuum for 17 hours, then at 80° C. under vacuum for 7 hours, giving a mixture of polymorphs A1 and E, primarily A1.

### Example 5i

### Preparation of Ladostigil Tartrate in 10 Volumes of Ethanol Containing 2% Dioxane as Co-Solvent

To the ethanolic solution A prepared as described in example 5f, dioxane 2% relative to ethanol weight was added and the solution was cooled in a controlled manner from 60° C. to 2° C. for 23 hours.

The wet material was determined to be polymorph C.

One part of the wet material was dried at 55° C. under vacuum for 17 hours, giving polymorph B.

Another part of the wet material was dried at 55° C. under vacuum for 17 hours, then at 80° C. under vacuum for 7 hours, giving a mixture of polymorphs A1 and E, primarily A1.

### Example 5j

### Re-Crystallization of Ladostigil Tartrate in 11 Volumes Acetone

Ladostigil tartrate (1.78 g) was dissolved in acetone (20 ml, 11 vol.) by heating the solution to 59° C. The mixture was seeded at 39° C. and cooled to 25° C. gradually. The product was collected by filtration.

The wet material was determined to be polymorph F with low crystallinity.

The wet material was dried at 80° C. under vacuum for 17 hours, giving polymorph A1.

### Example 5k

### Re-Crystallization of Ladostigil Tartrate in 43 Volumes Methylethyl Ketone

Ladostigil tartrate (0.46 g) is dissolved in methylethyl ketone (20 ml, 43 vol.) by heating the solution to 55° C. The mixture is seeded at 39° C. and cooled to 24° C. gradually. The product is collected by filtration.

The wet material was determined to be polymorph A1.

### Example 5L

### Re-Slurry in 32 Volumes Hot Iso-Butyl Acetate

Ladostigil tartrate polymorph A1 (1.26 g) was slurried in iso-butyl acetate (41 ml, 32 vol.) by heating the suspension at 62-70° C. The product was collected by filtration of the hot suspension.

The wet material was determined to be polymorph A1 with low crystallinity.

The wet material was dried at 50° C. under vacuum for 17 hours, giving polymorph A1.

### Example 5m

### Re-Slurry in 16 Volumes Hot Ethyl Acetate

Ladostigil tartrate polymorph A1 (0.18 g) was slurried in ethyl acetate (20 ml, 16 vol.) by heating the suspension at 62-70° C. The product was collected by filtration of the hot suspension.

The wet material was determined to be polymorph A1 with low crystallinity.

The wet material was dried at 50° C. under vacuum for 17 hours, giving polymorph A1.

### Example 5n

### Re-Slurry in 10 Volumes of Dioxane

Ethanol wet ladostigil tartrate polymorph C (20 g) was slurried in dioxane (200 ml) at 8-28° C. The product was collected by filtration.

The wet material was determined to be polymorph H.

The wet material dried at 80° C. under vacuum for 17 hours, giving polymorph A1.

### Example 5o

### Re-Crystallization of Ladostigil Tartrate in 8 Volumes of Ethanol Containing 2.5% Water as Co-Solvent

Ladostigil tartrate (2.5 g) is dissolved in a mixture of ethanol (20 ml) and water (0.5 ml) by warming the solution to 60° C. The mixture is self seeded at 41° C. and cooled to 25° C. gradually. The product is collected by filtration.

The wet material was determined to be polymorph C.

The material was dried at ambient temperature under vacuum for 17 hours, then at 80° C. under vacuum for 7 hours. It was determined to be polymorph A1.

### Example 5p

### Re-Crystallization of Ladostigil Tartrate in 8 Volumes of Ethanol Containing 0.2 Volumes of Water as a Co-Solvent

Ladostigil tartrate (18 g) was dissolved in a mixture of ethanol (144 ml) and water (3.6 ml) by warming the solution to 45° C. The mixture was seeded at 33° C. and cooled to 5° C. gradually over 6 hours. The product was collected by filtration and washed with 30 ml cold ethanol.

One part of the material was dried at ambient temperature under vacuum, giving polymorph C.

One part of the material was dried under vacuum at 80° C., giving a mixture of polymorph A1 and E, primarily form A1.

### Example 5q

### Re-Crystallization of Ladostigil Tartrate in 8 Volumes of Ethanol Containing 5% Acetic Acid as Co-Solvent

Ladostigil tartrate (2.5 g) was dissolved in a mixture of ethanol (20 ml) and acetic acid (0.5 ml) by warming the solution to 60° C. The mixture was cooled to 25° C. gradually. The product was collected by filtration.

The wet material was determined to be polymorph A1.

The wet material was dried at ambient temperature under vacuum for 17 hours, then at 80° C. under vacuum for 7 hours, giving polymorph A1.

### Example 5r

### Re-Crystallization of Ladostigil Tartrate in 8 Volumes of Ethanol Containing 7.5% Methanol as a Co-Solvent

Ladostigil tartrate (2.5 g) is dissolved in a mixture of ethanol (20 ml) and acetic acid (0.5 ml) by warming the solution to 60° C. The mixture was cooled to 25° C. gradually. The product was collected by filtration.

The wet material was determined to be a mixture of polymorph A1 and E, primarily A1.

The wet material was dried at ambient temperature under vacuum for 17 hours, then at 80° C. under vacuum for 7 hours, giving polymorph A1.

### Example 6

### Additional Methods for Preparing Polymorph A1

| Example | Procedure |
|---|---|
| 6-1 | Salt formation in EtOAc:EtOH (4:1) air drying at room |
| | temp |
| 6-2 | Salt formation in EtOAc:EtOH (8:1) air drying at room |
| | temp |
| 6-3 | Salt formation in EtOAc:EtOH (7:3) air drying at room |
| | temp |
| 6-4 | Salt formation in EtOAc:EtOH (3:1) air drying at room |
| | temp |
| 6-5 | Salt formation in EtOAc:EtOH (65:35) air drying at |
| | room temp |
| 6-6 | Salt formation in EtOAc:IPA:EtOH (8:1:1) air drying |
| | at room temp |
| 6-7 | Salt formation in EtOAc:IPA:EtOH (65:10:25) air |
| | drying at room temp |
| 6-8 | Salt formation in IPA air drying at room temp |
| 6-9 | Salt formation in EtOH:Diisopropyl ether (1:1) air |
| | drying at room temp |
| 6-10 | Salt formation in Acetone:EtOH (3:2) air drying at |
| | room temp |
| 6-11 | Salt formation in EtOAc:Acetone:EtOH (2:2:1) air |
| | drying at room temp |
| 6-12 | Salt formation in EtOAc:Acetone:EtOH (3:1:1) air |
| | drying at room temp |
| 6-13 | Salt formation in EtOAc:EtOH (85:15) air drying at |
| | room temp |
| 6-14 | Salt formation in EtOAc:EtOH (7:3) air drying at |
| | room temp |
| 6-15 | Salt formation in EtOAc:EtOH (55:45) air drying at |
| | room temp |
| 6-16 | Salt formation in EtOAc:Diisoprpyl ether:EtOH |
| | (3:1:1) air drying at room temp |
| 6-17 | Salt formation in EtOAc:Diisoprpyl ether:EtOH |
| | (2:2:1) air drying at room temp |
| 6-18 | Salt formation in EtOAc:Diisoprpyl ether:EtOH |
| | (5:1.1:1.1) air drying at room temp |
| 6-19 | Salt formation in Acetonitrile air drying at room |
| | temp |
| 6-20 | Salt formation in Acetonitrile:IPA (4:1) air drying |
| | at room temp |
| 6-21 | Salt formation in Acetonitrile:IPA (1:5) air drying |
| | at room temp |
| 6-22 | Salt formation in EtOH:IPA (1:3) air drying at |
| | room temp |
| 6-23 | Salt formation in EtOH:IPA (15:85) air drying at |
| | room temp |
| 6-24 | Salt formation in IPA:EtOH:EtOAc (4.2:1:1) air |
| | drying at room temp |
| 6-25 | Salt formation in IPA:EtOH:EtOAc (2:1:1) air drying |
| | at room temp |

### Example 7

### Re-Crystallization of Ladostigil Tartrate in Tetrahydrofuran

Ladostigil tartrate (1.55 g) was dissolved in tetrahydrofuran (20 ml, 13 vol.) by heating the solution to 60° C. The mixture was cooled gradually to 26° C. for 4.5 hours. The product was collected by filtration.

The wet material was determined to be polymorph A.

The material was dried under vacuum at ambient temperature for 14 hours, giving polymorph A1.

### Example 8

### Preparation of Ladostigil Tartrate in Ethanol (8.3 Volumes)

To a stirred solution of Ethyl-methyl-carbamic acid (R)-3-prop-2-ynylamino-indan-5-yl ester (74.6 g, 0.274 mole, 1 eq.) in absolute ethanol (335 ml, 265 gram) a warm solution of L-tartaric acid (21.6 g, 0.143 mole, 0.525 eq.) in absolute ethanol (158 ml, 125 gram) was added. The residue of tartaric acid in the vessel was washed with 126 ml (100 gram) absolute ethanol into the reaction mixture. The mixture was heated to 58-62° C. and filtered. The hot solution was divided in to four parts and returned to four reactors with different stirring devices (impeller and buffle), seeded at 37° C. and cooled to 0-5° C. gradually for 18 hours. The products from each reactor were collected by filtration and washed with cold ethanol.

The wet material was determined to be polymorph C. Some of the samples contained polymorph C with low crystallinity. The wet material was dried at 55° C. under vacuum for 17 hours to give polymorph E. The wet polymorph with low crystallinity was transformed upon drying under the same conditions to polymorph E with low crystallinity. Both polymorph E and polymorph E with low crystallinity were transformed to form A upon standing.

Further drying of materials polymorph E under vacuum at 80° C. for 7 hours gave the mixture of polymorphs A1 and E, with primarily polymorph A1.

### Example 9

### Preparation of Ladostigil Tartrate in Ethanol (12 Volumes)

The same procedure as in Example 8 was repeated with a larger amount of ethanol.

The wet material was determined to be polymorph C.

The wet material was dried at 80° C. under vacuum for 17 hours and polymorph E was formed. Polymorph E was transformed to polymorph A upon standing.

### Example 10

### TGA and Crystal Form of Ladostigil Tartrate form A1 and Various Humidity Conditions

Ladostigil tartrate form A1 was exposed to various humidity conditions at room temperature for 14 days. TGA was performed, and crystal form was analyzed. The results are summarized in table 3.

TABLE 3

| RH [%] | TGA | Resulting form |
|---|---|---|
| 60 | 0.2 | A1 |
| 80 | 0.1 | A1 |
| 100 | 0.1 | A1 + A |

It is evident from table 3 that pure form A1 was transformed to a mixture of Form A and A1 after exposure to 100% RH for 14 days, but the water content did not change significantly.

From these results it appears that neither Form A nor Form A1 is hygroscopic.

### Example 11

### Stability of Mixture of Ladostigil Tartrate Mixture of Form A1 and Form E at High Humidity

Crystal form of ladostigil tartrate form A1>E exposed at various humidity conditions at room temperature for 14 days was determined. The results are summarized in table 4.

TABLE 4

| RH | Form |
|---|---|
| 60 | A1 > E |
| 80 | A1 > E |
| 100 | A1 > A |

Table 4 illustrates that when a mixture of form A1 and form E was exposed to 100% relative humidity, the mixture was transformed to a mixture of form A1 and A, primarily form A1.

### Example 12

### Effect of Heating

Various polymorph forms and mixtures thereof were heated. The heating conditions and resulting polymorph forms are described in table 5 below:

TABLE 5

| Starting | | Time | |
|---|---|---|---|
| form | Experimental conditions | (hours) | Resulting form |
| A1 + A | RT under vacuum | | A1 + A |
| | 55° C. under vacuum | 17 | A1 + A |
| A1 | RT under vacuum | | A1 |
| | 50° C. under vacuum | 12 | A1 |
| | 55° C. | 17 | A1 |
| | RT for 17 hours, then 80° C. | 7 | A1 |
| | 80° C. | | A1 |
| B | 80° C. | 6 | A1 + E |
| | 80° C. | 9 | A1 + E |
| | 80° C. | 17 | A1 + E |
| C | 50° C. under vacuum | 12 | B |
| | 55° C. | 17 | B |
| | 55° C. | 19 | B |
| | 55° C. under vacuum | 17 | E |
| | 50° C. under vacuum | 1 | A1 + C |
| | 50° C. under vacuum for 20 | 4 | A1 + E |
| | hours then 80° C. | | |
| | 50° C. under vacuum for 12 | 3 | A1 + E |
| | hours then 80° C. | | |
| F | 80° C. under vacuum | | A1 + A |
| | 80° C. under vacuum | | A1 |
| H | 80° C. under vacuum | | A1 + A |
| | 80° C. under vacuum | | A1 |

Form A+A1 (30 and 80% Form A content), when dried at 55 ° C., retained the same polymorphic content of A+A1.

At 80° C., Form B transformed to a mixture of Form A1 and form E, primarily form A1. There were no significant differences in form E concentration between the samples dried for 6 or 9 or 17 hours at 80° C.

Form C transformed to different mixtures of A1, B, C, E as a function of drying temperature and drying time. After 1 hour of drying at 50° C., Form C was still detected. After drying for 12 hours at 50° C. under vacuum, or for 17-19 hours at 55° C., Form B was formed. After drying for 17 hours at 55° C. under vacuum Form E was obtained.

Forms F and H both transformed to Form A1 and A at 80° C. under vacuum.

### Example 13

### Effect of Micronization, Pressing and Grinding

The effects of micronization, pressing (1 minute at 100 metric tons) and grinding were determined by XRD and the following results were obtained:

Form A transformed to a mixture of Forms A and A1 upon grinding, and after pressing form A transformed mainly to Form A1. Crystallinity degradation was also observed upon pressing. Form A and Form A1 lose some extent of crystallinity after pressing. The XRD peaks become broader and the intensities became smaller after grinding and pressing the sample.

### Example 14

### Preparation of Form F by Saturated Atmosphere DMF/Hexane

A saturated solution of ladostigil tartrate (conc. 0.35 g/ml) in dimethylformamide was placed in a beaker that is contained in a chamber saturated with n-hexane vapors. The solution was kept in the chamber for 7 days. The crystals which formed in the beaker were collected and dried under vacuum without heating until a constant weight was achieved. The crystals were determined to be form F.

### Example 15

### Preparation of Form H by Re-Slurry in 10 Volumes of Dioxane

Wet ladostigil tartrate polymorph C (20 g) was slurried in dioxane (200 ml) at 8-28° C. The product was collected by filtration.

The wet material was determined to be polymorph H.

The wet material dried at 80° C. under vacuum for 17 hours, giving polymorph A1.

What is claimed is:

1. A process for preparing crystalline ladostigil tartrate characterized by an x-ray diffraction pattern having peaks at 8.7, 13.9, and 17.4±0.2 degrees two theta, comprising the steps of:
(a) preparing a solution of ethyl-methyl-carbamic acid (R)-3-prop-2-ynylamino-indan-5-yl ester in a solvent selected from the group consisting of ethanol, ethyl acetate, acetone, acetonitrile, diisopropylether and mixtures thereof;
(b) combining tartaric acid with the solution to form a precipitate;
(c) recovering the precipitate; and
(d) drying the precipitate to obtain the crystalline ladostigil tartrate.

2. The process of claim 1, wherein the solution further comprises at least one of toluene or dioxane.

3. The process of claim 1, wherein the solution of step b) is heated at about reflux temperature or lower.

4. The process of claim 1, wherein the precipitate is formed by cooling at a temperature of about 30° C. to about 0° C.

5. The process of claim 1, wherein the precipitate is dried under a pressure of less than about 100 mm Hg.

6. The process of claim 5, wherein the precipitate is dried at a temperature of about 50° C. to about 90° C.

7. The process of claim 1, wherein the solvent is ethanol.

8. The process of claim 1, wherein the solvent is ethyl acetate.

9. The process of claim 1, wherein the solvent is acetone.

10. The process of claim 1, wherein the solvent is acetonitrile.

11. The process of claim 1, wherein the solvent is diisopropylether.

12. A process for preparing crystalline ladostigil tartrate characterized by an x-ray diffraction pattern having peaks at 8.7, 13.9, and 17.4±0.2 degrees two theta, comprising the steps of:
(a) preparing a solution of ladostigil tartrate in a C₁-C₄ alcohol, acetone, methyl ethyl ketone, tetrahydrofuran, acetonitrile, ethyl acetate, diisopropylether or mixtures thereof;
(b) precipitating the crystalline ladostigil tartrate; and
(c) recovering the crystalline ladostigil tartrate.

13. The process of claim 12, wherein the solution further comprises water or acetic acid.

14. The process of claim 12, wherein the solution is heated at about reflux temperature or lower.

15. The process of claim 12, wherein step b) comprises cooling the solution at a temperature of about 30° C. to about 0° C.

16. The process of claim 12, wherein step b) comprises seeding the solution.

17. The process of claim 12, wherein the crystalline ladostigil tartrate is dried under a pressure of less than about 100 mm Hg.

18. The process of claim 17, wherein the crystalline ladostigil tartrate is dried at a temperature of about 50° C. to about 90° C.

19. The process of claim 12, wherein the solvent is a C₁-C₄ alcohol.

20. The process of claim 12, wherein the solvent is acetone.

21. The process of claim 12, wherein the solvent is methylethylketone.

22. The process of claim 12, wherein the solvent is tetrahydrofuran.

23. The process of claim 12, wherein the solvent is acetonitrile.

24. The process of claim 12, wherein the solvent is ethyl acetate.

25. The process of claim 12, wherein the solvent is diisopropylether.

26. A process for preparing crystalline ladostigil tartrate characterized by an x-ray diffraction pattern having peaks at 8.7, 13.9, and 17.4±0.2 degrees two theta, comprising the steps of:
(a) maintaining a heterogeneous mixture of ladostigil tartrate in acetate, ethyl acetate, dioxane or mixtures thereof; and
(b) recovering from the mixture the crystalline ladostigil tartrate.

27. The process of claim 26, wherein the mixture of step a) is heated at a temperature of about 50° C. to about 80° C.

28. The process of claim 26, wherein the crystalline ladostigil tartrate is recovered by drying under a pressure of less than about 100 mm Hg.

29. The process of claim 28, wherein the crystalline ladostigil tartrate is dried at a temperature of about 50° C. to about 90° C.

30. A process for preparing crystalline ladostigil tartrate characterized by an x-ray diffraction pattern having peaks at 8.7, 13.9, and 17.4±0.2 degrees two theta, comprising drying ladostigil tartrate form B or form C.

31. The process of claim 30, wherein the ladostigil tartrate is dried under a pressure of less than about 100 mm Hg.

32. The process of claim 31, wherein the ladostigil tartrate is dried at a temperature of about 50° C. to about 90° C.

33. A process for preparing crystalline ladostigil tartrate characterized by an x-ray diffraction pattern having peaks at 8.7, 13.9, and 17.4±0.2 degrees two theta, comprising heating ladostigil tartrate form B, form C, form F, or form H.

34. The process of claim 33, wherein the ladostigil tartrate form B, form C, form F, or form H is heated under a pressure of less than about 100 mm Hg.

35. The process of claim 34, wherein the ladostigil tartrate form B, form C, form F, or form H is heated at a temperature of about 40° C. to about 90° C.

36. A pharmaceutical composition comprising a therapeutically effective amount of crystalline ladostigil tartrate characterized by an x-ray diffraction pattern having peaks at 8.7, 13.9, and 17.4±0.2 degrees two theta, and a pharmaceutically acceptable carrier.

37. The pharmaceutical composition of claim 36, wherein the crystalline ladostigil tartrate is prepared according to any one of claims 1, 12, 26, 30, or 33.

38. A process of preparing a pharmaceutical composition comprising the step of combining crystalline ladostigil tartrate characterized by an x-ray diffraction pattern having peaks at 8.7, 13.9, and 17.4±0.2 degrees two theta, or a solution prepared from crystalline ladostigil tartrate characterized by an x-ray diffraction pattern having peaks at 8.7, 13.9, and 17.4±0.2 degrees two theta, with a pharmaceutically acceptable carrier.

39. A method of treating Alzheimer's disease comprising administering to a human subject in need thereof the pharmaceutical composition of claim 36.

40. A method of treating a mammal in need of inhibition of the acetylcholine esterase enzyme comprising administering the pharmaceutical composition of claim 36 to the mammal.

41. A method of treating a mammal in need of inhibition of the monoamine oxidase type B enzyme comprising administering the pharmaceutical composition of claim 36 to the mammal.
